(19) United States
(12) Patent Application Publication
(10) Pub. No.: US 2018/0028147 A1
(43) Pub. Date: Feb. 1, 2018

(54) AUTO FINGER JOINT DETECTION FOR ROBOTIC HAND ULTRASOUND SCANNER

(71) Applicant: General Electric Company, Schenectady, NY (US)

(72) Inventors: Dongqing CHEN, New Berlin, WI (US); Menachem HALMANN, Wauwatosa, WI (US); Xiaodong HAN, Shanghai (CN); Hong CHENG, Shanghai (CN)

(21) Appl. No.: 15/219,712

(22) Filed: Jul. 26, 2016

(51) Int. Cl. A61B 8/08; A61B 5/107; A61B 8/00; A61B 5/00

(52) U.S. Cl. CPC A61B 8/0875; A61B 5/4887; A61B 5/4528; A61B 5/4538; A61B 8/5215; A61B 5/0082; A61B 5/1072; A61B 8/4483; A61B 8/4281

(57) ABSTRACT

Methods of determining a location of a desired joint in a scanning assembly are provided. The method comprises acquiring a digital image of an extremity against a background within the scanning assembly. The extremity has a plurality of digits extending away from a base of the extremity and the plurality of digits has a plurality of corresponding joints. The method also comprises extracting the outline of at least a portion of the extremity, locating a midpoint along a width of a base of the extremity, identifying a plurality of clusters of texture features; and, determining the location of a desired joint based on a distance between the center point and the midpoint.

### CROSS REFERENCE TO RELATED APPLICATIONS

This application is a continuation-in-part of a prior filed, co-pending U.S. patent application Ser. No. 14/541,827 (GE Docket No. 275036-1), filed on Nov. 14, 2014, titled “Finger Joint Ultrasound Imaging”, the contents of which is herein incorporated in its entirety.

### BACKGROUND OF THE INVENTION

Rheumatoid arthritis (RA) is a type of inflammatory arthritis that affects 1.3 million Americans and globally impacts 22 million patients with annual growth rate at 1 to 3%. Advances in treatment and monitoring (including ultrasound) have resulted in patients achieving early and sustained clinical and imaging resolution.

Ultrasound imaging is used by healthcare providers to diagnose medical conditions and to determine treatment for a patient. In many circumstances, it is beneficial to use ultrasound imaging to obtain images of various parts of the body. More particularly, ultrasound images of the hand and foot joints are beneficial when diagnosing injuries or when such joints become inflicted with a disease such as rheumatoid arthritis. However, with current methods (i.e. x-ray), it is often difficult to capture images of such joints in a consistent and efficient manner. For example, current methods take a significant amount of time to locate a desired joint for patients inflicted with a disease such as rheumatoid arthritis due to the painful swelling that patients experience during the testing process.

Therefore, there is a need for methods that ease the work of healthcare providers and increase the comfort level of patients when using ultrasound imaging to determine a location of a desired joint for the monitoring and diagnosis of an ailment such as rheumatoid arthritis.

### SUMMARY OF THE INVENTION

Embodiments of the present invention seek to provide an improved method for determining a location of a desired joint in a scanning assembly that is faster and more efficient, which will benefit healthcare providers and patients. Benefits and advantages of the methods provided herein include improved patient experience, non-invasive procedures, and easy to use systems that produce real-time images. Various other benefits will also become apparent from the description that follows.

In one embodiment, the method includes acquiring a digital image of an extremity against a background within the scanning assembly. More particularly, the scanning assembly is an ultrasound scanning assembly. The extremity includes a plurality of digits extending away from the base of the extremity. The plurality of digits includes a plurality of corresponding joints. The method also includes extracting the outline of at least a portion of the extremity; locating a midpoint along a width of a base of the extremity; identifying a plurality of clusters of texture features, each having a corresponding center point; and determining the location of a desired joint based on a distance between the center point and the midpoint. The step of locating a midpoint along a width of a base of the extremity may be determined by measuring a width of a human wrist.

In another embodiment, a method is provided for determining a location of a desired joint in a scanning assembly. The method includes acquiring a digital image of an extremity against a background within the scanning assembly. The extremity having a plurality of digits extending away from a base of the extremity, the plurality of digits having a plurality of corresponding joints; extracting the outline of at least a portion of the extremity; plotting a curve of at least one of the plurality of peaks against at least one of the corresponding plurality of valleys; averaging a distance between at least two adjacent valleys on the curve in order to identify the position of a desired joint.

In yet another embodiment, the extremity may be a hand or a foot; and at least one of the plurality of digits is one of a finger or a toe. In yet another embodiment, the plurality of peaks may correspond to fingertips of the hand, and the corresponding plurality of valleys may correspond to finger valleys of the hand. More particularly, the step of acquiring a digital image may include utilizing an optical camera. In an aspect, the width of the base is determined by measuring a width of a human wrist. In another aspect, the step of identifying the cluster of texture features further comprises performing a k-means clustering calculation to isolate the desired joint. In a fourth aspect, determining the location of a desired joint, i.e. a human PIP joint, is performed automatically.

### BRIEF DESCRIPTION OF THE DRAWINGS

Various aspects and embodiments of the present innovation will now be described in connection with the accompanying drawings, in which:

FIG. 1 is a schematic diagram of an example ultrasound scanning assembly, specifically for identifying joints on a human finger.

FIG. 2 is a flow diagram of an example method for locating a desired joint using k-means clustering.

FIGS. 3A, 3B, 3C and 3D illustrate the use of k-mean clustering to locate a desired joint as described herein.

FIG. 4 is a flow diagram of another example method for acquiring ultrasound images of a joint using peak-valley points.

FIGS. 5A, 5B and 5C illustrates the use of peak-valley points to locate a desired joint as described herein.

### DETAILED DESCRIPTION

The objects and advantages enumerated above together with other aspects, features, and advances represented by the present invention will now be presented in terms of detailed embodiments described with reference to the attached drawing figures which are intended to be representative of various possible configurations of the invention. Examples illustrated herein are directed to an example in which a human hand is being scanned for assistance with treatment of RA or other inflammatory diseases. Other applications of the systems and methods of the invention are envisioned such as diagnosing or treating other injuries or diseases of the human foot or corresponding animal extremities.

In one aspect, the finger joint may be the proximal interphalangeal joint (PIP Joint). In this aspect, the method of determining a location of a desired joint in a scanning assembly, utilizes an algorithm (i.e., k-means clustering) for the PIP joints. The PIP joints are the middle joints of the fingers. They are shaped as double domes side by side on the hand side of the joint and two side by side matching shallow curves on the finger side. This allows mobility in bending and straightening but stability in sideways and twisting movements. The PIP joints are therefore very stable but this makes them more prone to stiffness and other problems, such as RA.

In another aspect, the finger joint may be the metacarpophalangeal joint (MCP Joint). In this aspect, the method of determining a location of a desired joint in a scanning assembly, utilizes peaks valley points searching for the MCP joints. The MCP joints are the largest joints of the hand and are important for both power grip and pinch activities. The MCP joints are often affected by RA either from routine wear and tear, an injury, or medical conditions. Specifically, RA affects the inner coating of the joint, called the synovium, and can result in the loss of the cartilage between the joints. Cartilage is the coating layer of tissue on the end of a bone that acts as a shock absorber. Loss of cartilage can lead to joint destruction and a shift in the finger position towards the small finger side.

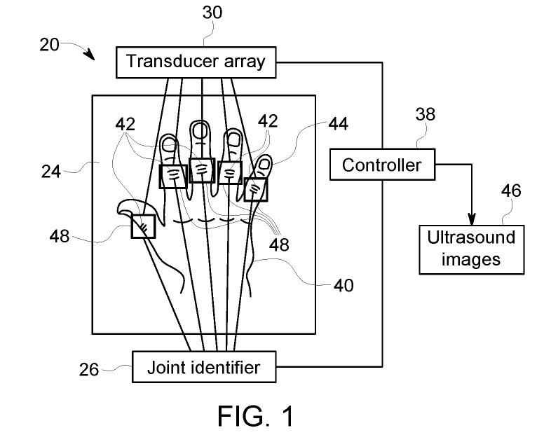

With reference now to the drawing figures, and first to FIG. 1. FIG. 1 schematically illustrates a scanning assembly, more particularly an ultrasound imaging system 20. As described hereafter, an ultrasound imaging system 20 consistently captures images of joints in an efficient manner. In the example illustrated, the ultrasound imaging system 20 comprises an ultrasound scanning assembly comprising fluid 24, transducer array 30, joint location identifier 34 and controller 38.

Fluid 24 comprise a volume of fluid which serves as an acoustic coupling between a person's extremity 40, such as a hand, and transducer array 30. In one implementation, fluid 24 comprises a bath of water in which the extremity 40 is immersed during scanning by transducer array 30 of imaging system 20. In other implementations, fluid 24 may comprise other forms of a fluid, such as other forms of a liquid, gel or the like, which serve as an acoustic coupling between the extremity 40 and transducer array 30.

Transducer array 30 comprises an array of transducers that output signals to facilitate the acquisition of ultrasound images of the joints of the extremity 40. In the example illustrated, transducer array 30 comprises crystals, such as quartz crystals or piezoelectric crystals, which change shape in response to the application of electrical current so as to produce vibrations or sound waves. Likewise, the impact of sound or pressure waves upon such crystals produce electrical currents. As a result, such crystals are used to send and receive sound waves. Each of the transducers of transducer array 30 may additionally include a sound absorbing substance to eliminate back reflections and an acoustic lens to focus emitted sound waves.

Joint location identifier 34 comprises a device by which locations of a joint 42 or multiple joints of an extremity 40 are provided to controller 38. In the example illustrated, joint location identifier 34 identifies the location of the joints 42 on the digits 44 which in the example are the second knuckles of each of the digits 44 of the extremity 40 relative to the base of the extremity. Digits 44 may correspond to fingers as shown, but may also be toes or portions of human or animal phalanges sought to be scanned and tested. For purposes of this disclosure, the term “fingers” includes a person's thumb as well as the remaining digits of a hand. As will be described hereafter, the identified locations of joints 42 are used by controller to control the operation and/or positioning of transducer array 30.

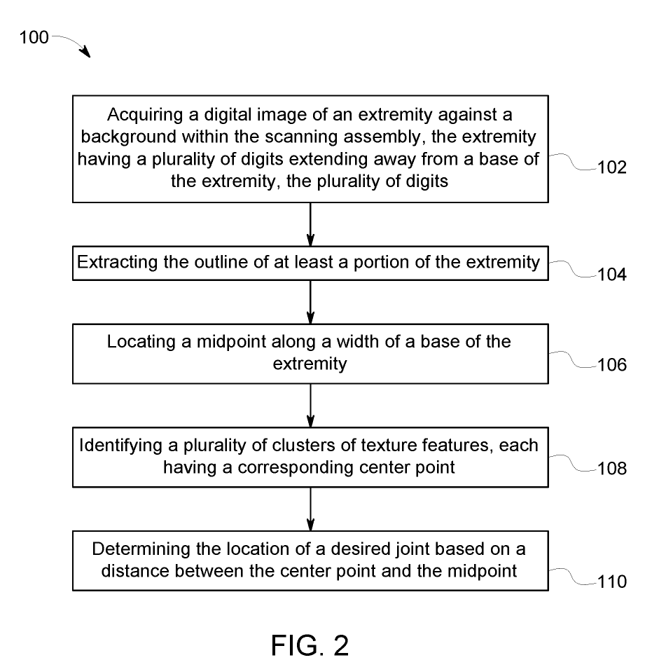

FIG. 2 is a flow diagram of an example method 100 for determining a location of a desired joint in a scanning assembly. In one implementation, method 100 may be carried out by system 20 of FIG. 1. In other implementations, method 100 may be carried out by other scanning assemblies.

As indicated by block 102 in FIG. 2, method 100 comprises acquiring a digital image of an extremity 40, in a scanning assembly 20, such as an imaging system, including a transducer array 30 and a fluid 24 providing an acoustic coupling between the transducer array 30 and the extremity 40. The extremity 40 has a plurality of digits 44 extending away from a base of the extremity 40, and the plurality of digits 44 have a plurality of corresponding joints. In another embodiment, the step of acquiring a digital image may include utilizing an optical camera within the scanning assembly 20.

As indicated by block 104 of FIG. 2, method 100 further comprises a step of extracting the outline of at least a portion of the extremity 40 identifying locations of a plurality of joints 42, of the extremity 40 while the extremity 40 is stationary in the scanning assembly 20.

As indicated by block 106 of FIG. 2, method 100 comprises the step of locating a midpoint along a width of a base of the extremity 40 acquiring ultrasound images of the plurality of joints 42 with the transducer array 30 while the extremity 40 is held stationary in the imaging system 20, wherein the joint focused images 46 are of an area less than the entire area of the extremity 40 based upon the identified locations of the joints 42 of the extremity 40. In an embodiment, the width of the base of an extremity 40 may be determined by measuring a width of a human wrist.

As indicated by block 108 of FIG. 2, method 100 comprises the step of identifying a plurality of clusters of texture features, wherein each having a corresponding center point of the extremity 40 identifying locations of a plurality of joints 42, of the extremity 40. Texture features refer to the wrinkles or areas of gathered, and/or hyper-pigmented skin that can be seen on the surface of a joint. In an embodiment, the step 108 also includes performing k-means clustering calculations to isolate the desired joint, as will be discussed below.

As indicated by block 110 of FIG. 2, method 100 further comprises a step of determining the location of a desired joint of the extremity 40 based on a distance between the center point and the midpoint of the extremity 40 identifying locations of a plurality of joints 42, of the extremity 40 while the extremity is stationary in the scanning assembly 20. In an embodiment, determining the location of a desired joint is performed automatically.

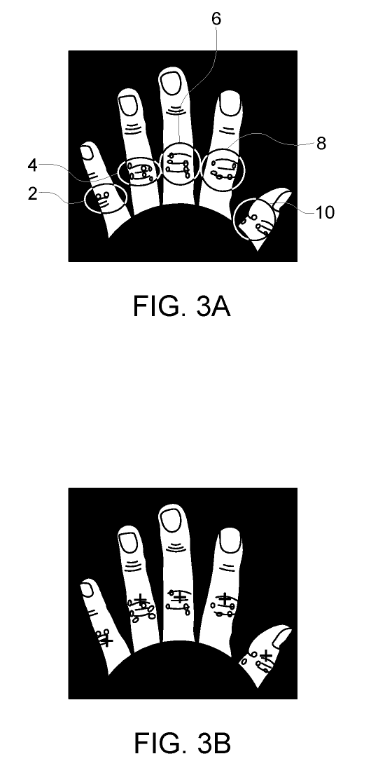

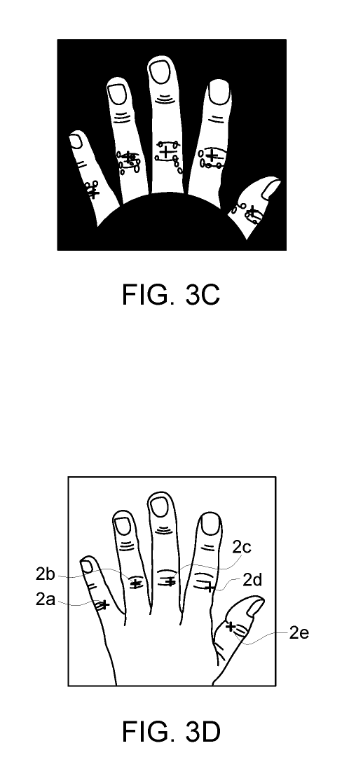

FIGS. 3A, 3B, 3C and 3D illustrate locating a region of interest on at least one PIP joint on a human hand. Particularly FIGS. 3A-3D illustrates how to perform k-means clustering to determine a specific centers of regions of interest 2a-2e on the desired joint or joints. While all five centers 2a-2e are located in this example, one of ordinary skill would appreciate the method described herein can be used to describe any number of centers under examination.

Turning to FIG. 3A, five groups of cloud points 2, 4, 6, 8, and 10 are circled around the five PIP joints. The five initial seed points are initialized, one for each group as shown in FIG. 3B. Next the distance between each cloud point 2-10 to each seed point is calculated, where the distance is the Euclidean distance between each cloud point in 2-10 and each seed point (initial centroid). Then based on the minimum distance of each cloud point to the five seed points, the cloud points 2-10 are regrouped to five new groups, and calculate five centroids of five groups of cloud points as the new seed points, as shown in FIG. 3C. The steps calculating the distances and regrouping the cloud points are repeated, until the distance between updated and old centroid for each group do not change. Eventually, the five centroids will be considered as the center of region of interest 2a-2e around each PIP Joint, as shown in FIG. 3D.

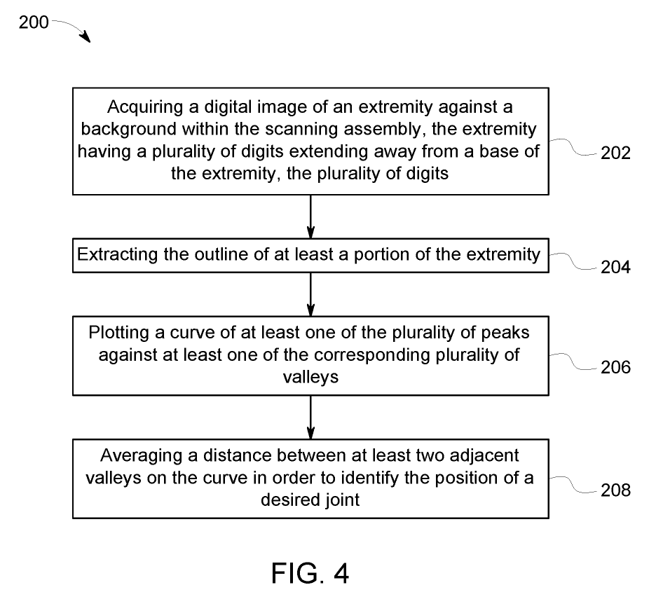

FIG. 4 is a flow diagram of an example method 200 for determining a location of a desired joint in a scanning assembly. In one implementation, method 200 may be carried out by system 20 of FIG. 1. In other implementations, method 100 may be carried out by other scanning assemblies.

As indicated by block 202, method 200 comprises acquiring a digital image of an extremity 40 in a scanning assembly 20, such as an imaging system, including a transducer array 30 and a fluid 24 providing an acoustic coupling between the transducer array 30 and the extremity 40. The extremity 40, has a plurality of digits 44 extending away from a base of the extremity, and the plurality of digits 44 have a plurality of corresponding joints. The plurality of digits 44 may be a finger or a toe.

As indicated by block 204 of FIG. 4, method 200 further comprises the step of extracting the outline of at least a portion of the extremity 40 and identifying locations of a plurality of joints 42 of the extremity 40 while it is stationary within the scanning assembly 20.

Step 206 includes plotting a curve of at least one of the plurality of peaks corresponding to edges against at least one of the corresponding plurality of valleys of the extremity 40, defining the digits 44. Ultrasound images of the plurality of joints 42 are acquired by the transducer array 30 while the extremity 40 is held stationary in the imaging system 20. In an embodiment, images 46 are focused in on the desired joints, and acquired of an area less than the entire area of the extremity 40 based upon the identified locations of the desired joints 42 of the extremity 40. In an embodiment, the width of the base of the extremity 40 may be determined by measuring a width of a human wrist or base of foot at or near the ankle. In another embodiment, the plurality of peaks corresponds to fingertips of the hand, and the corresponding plurality of valleys corresponds to finger valleys of the hand.

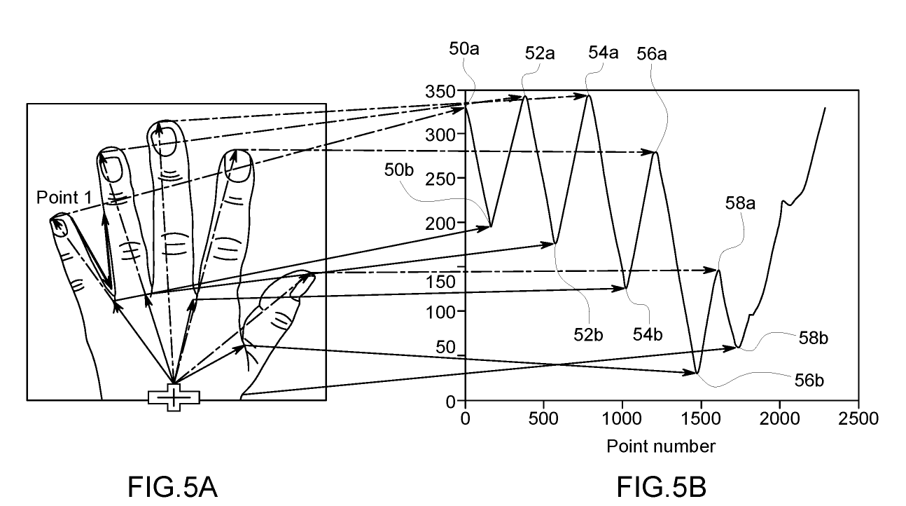

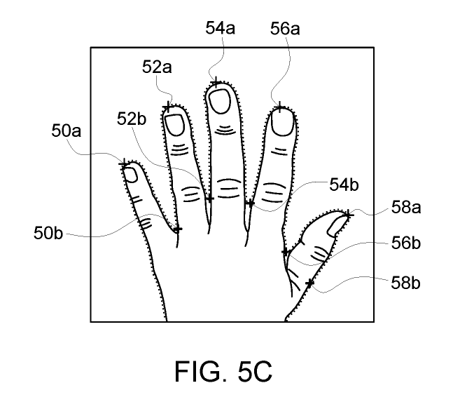

FIGS. 5A, 5B and 5C illustrate how to perform peak-valley points search for the desired joint, more particularly in a MCP joint according to embodiments of the invention. As shown in FIG. 5A, the distance is calculated between the marked center point and each hand profile boundary point, e.g. point 1, set as the finger tip of small finger. FIG. 5B shows the horizontal axis as the point number, e.g., around 2300 hand boundary points, and the vertical axis as the distance between each of around 2300 boundary points to the marked center point, which is plotted on the distance map. The local maximum points 50a-58a and minimum points 50b-58b of the curve in FIG. 5B are determined. All of the local maximum and minimum points will be found using the differentiation of the curve. The local maximum 50a-58a and minimum points 50b-58b correspond to the finger-tip and valley points linked using different lines between FIGS. 5A and 5B. FIG. 5C shows the final results of the finger-tip 50a-58a and valley points 50b-58b.

As indicated by block 208 of FIG. 4, method 200 further comprises a step of averaging a distance between at least two adjacent valleys, e.g., 50b and 52b, on the curve in order to identify the position of a desired joint, of the extremity 40. The centers of the five PIP joints are the five final centroids of the five cloud wrinkle points, which are calculated in [0028], and the center of the five MCP joints are located by finding the middle point location of two adjacent valley points, e.g., the ring finger MCP center position is calculated by finding the middle point of the two adjacent valleys, 50b and 52b. Once the center of PIP or MCP joints are located, the ultrasound probe will move and cover the Region of Interest (ROI) of PIP or MCP by aligning its center and the center of PIP or MCP joint together.

An advantage of various aspects described herein is a significant reduction in the scanning time when locating the desired joints. In diagnosing or treating patients with RA the desired joint.

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of determining a location of a desired joint in a scanning assembly, the method comprising:
acquiring a digital image of an extremity against a background within the scanning assembly, the extremity having a plurality of digits extending away from a base of the extremity, the plurality of digits having a plurality of corresponding joints;
extracting the outline of at least a portion of the extremity;
locating a midpoint along a width of a base of the extremity;
identifying a plurality of clusters of texture features, each having a corresponding center point;
determining the location of a desired joint based on a distance between the center point and the midpoint.

2. The method of claim 1, wherein the extremity is one of a hand, and a foot; and at least one of the plurality of digits is one of a finger and a toe.

3. The method of claim 1, wherein the scanning assembly is an ultrasound scanning assembly.

4. The method of claim 1, wherein the step of acquiring a digital image includes utilizing an optical camera.

5. The method of claim 2, wherein the width of the base is determined by measuring a width of a human wrist.

6. The method of claim 1, wherein the step of identifying the at least one cluster of texture features further comprises performing a k-means clustering calculation to isolate the desired joint.

7. The method of claim 1, wherein the step of determining the location of a desired joint is performed automatically.

8. The method of claim 2, wherein the desired joint is a human PIP joint.

9. A method of determining a location of a desired joint in a scanning assembly, the method comprising:
acquiring a digital image of an extremity against a background within the scanning assembly, the extremity having a plurality of digits extending away from a base of the extremity, the plurality of digits having a plurality of corresponding joints;
extracting the outline of at least a portion of the extremity;
plotting a curve of at least one of the plurality of peaks against at least one of a corresponding plurality of valleys formed between at least two of the plurality digits;
averaging a distance between at least two adjacent valleys on the curve in order to identify the position of a desired joint.

10. The method of claim 9, wherein the extremity is one of a hand, and a foot; and at least one of the plurality of digits is one of a finger and a toe.

11. The method of claim 9, wherein the scanning assembly is an ultrasound scanning assembly.

12. The method of claim 9, wherein the step of acquiring a digital image includes utilizing an optical camera.

13. The method of claim 9, wherein the plurality of peaks corresponds to fingertips of the hand, and the corresponding plurality of valleys corresponds to finger valleys of the hand.

14. The method of claim 9, further comprising the step of, locating a midpoint along a width of a base of the extremity.

15. The method of claim 14, wherein the width of the base is determined by measuring a width of a human wrist.
